(19) United States
(12) Patent Application Publication
(10) Pub. No.: US 2012/0058095 A1
(43) Pub. Date: Mar. 8, 2012

(54) OILY SUSPENSION CONTAINING PROBIOTIC BACTERIA FOR PAEDIATRIC USES

(75) Inventors: Gian Paolo Strozzi, Novara (IT); Luca Mogna, Novara (IT)

(73) Assignee: PROBIOTICAL S.p.A., Novara (IT)

(21) Appl. No.: 13/255,176

(22) PCT Filed: Mar. 9, 2010
(86) PCT No.: PCT/IB2010/000486

§ 371 Date: Nov. 21, 2011

(30) Foreign Application Priority Data
Mar. 9, 2009 (IT) RM2009A000104

(51) Int. Cl. A61K 35/74; A61P 1/00

(52) U.S. Cl. 424/93.44; 424/93.4; 424/93.45

(57) ABSTRACT

The present invention refers to an oily suspension containing probiotic bacteria, particularly suitable for paediatric use.

The present invention refers to an oily suspension containing probiotic bacteria, particularly suitable for paediatric use.

It is well-known that there are on the market liquid compositions containing an oil and lactic bacteria for the treatment of a number of intestinal disturbances such as, for example, colic in paediatric patients.

Said compositions, however, have a number of disadvantages which limit their use.

A first disadvantage relates to the instability of the bacteria inside the composition.

In practice, lactic bacteria immersed in oil suffer from a lack of stability which causes a decay in the bacterial load over time.

The initial declared bacterial load for a given composition decays over time because of the lack of stability of the bacteria within it.

Therefore, the initial bacterial load present in the starting product does not correspond, after a certain relatively brief lapse of time after the time of manufacture, to what is stated in the declaration on the label, because of the decay which occurs in the bacterial load.

A second disadvantage relates to the nature of the oil which not only affects the state of vitality of the bacteria but can condition their efficacy, once administered within the organism (in-vivo vitality and functionality).

Finally, there are also some considerations to make on the stability of the composition itself. In practice, after a certain interval of time lactic bacteria immersed in oil can give rise to precipitations or aggregations, with the subsequent formation of a sediment. These phenomena can alter the “shelf life” of the composition.

The necessity therefore remains of having available a liquid composition containing an oil and probiotic bacteria, having an improved stability by comparison with the compositions presently available on the market. In practice, it is important to prepare a composition containing an oil and probiotic bacteria in which the bacterial load initially present is not subject to excessive decay over time leading to a drastic reduction in said bacterial load. Finally, it is necessary for the composition containing an oil and probiotic lactic bacteria to be prepared so as to maintain the bacteria in a good state of vitality and functionality.

The Applicant has responded to the above-mentioned needs by perfecting an oily suspension comprising at least one oil, edible and utilizable in human nutrition, and at least one probiotic micro-organism, as stated in the attached independent claim.

The suspension which is the subject of the present invention has applications in patients of paediatric age, as stated in the attached independent claim.

In particular, the Applicant has perfected a supplement having a composition which comprises a food matrix and probiotic micro-organisms.

Embodiments of the present invention are set forth in the detailed description which follows, in exemplary form and therefore not limiting the range. of the present invention.

Probiotic micro-organisms are live bacteria capable of producing a beneficial effect on the consumer when ingested in sufficient quantities and for a sufficient time.

Probiotics usually belong to the genera Lactobacillus, Bifidobacterium, Streptococcus, Lactococcus, Pediococcus, Propionibacterium, Leuconostoc and Saccharomyces.

In the sphere of lactic bacteria of the genus Lactobacillus, the species endowed with probiotic activity are L. acidophilus, L. crispatus, L. gasseri, L. delbrueckii group, L. salivarius, L. casei, L. paracasei, L. plantarum group, L. rhamnosus, L. reuteri, L. brevis, L. buchneri, L. fermentum, L. fructivorans, L. ruminis, L. sakei and L. vaginalis.

Among the other lactic bacteria we recall Streptococcus thermophilus, Pediococcus pentosaceous, Leuconostoc argentinum and mesenteroides, while the following belong to the genus Bifidobacterium: B. adolescentis, B. angulatum, B. bifidum, B. breve, B. catenulatum, B. infantis, B. lactis, B. longum and B. pseudocatenulatum.

The probiotics used in the preparation of the oily suspension in accordance with the present invention are selected from the group comprising the following species: L. acidophilus, L. crispatus, L. gasseri, L. delbrueckii group, L. salivarius, L. casei, L. paracasei, L. plantarum group, L. rhamnosus, L. reuteri, L. brevis, L. buchneri, L. fermentum, B. adolescentis, B. angulatum, B. bifidum, B. breve, B. catenulatum, B. infantis, B. lactis, B. longum, B. pseudocatenulatum and S. thermophilus.

In a preferred embodiment of the invention, the suspension comprises from one to six strains, preferably from two to four strains, even more preferably three strains selected from among the probiotic species mentioned above.

Table 1 shows, by way of example, a group of micro-organisms which have valid application in the context of the present invention.

All the strains have been deposited in accordance with the Treaty of Budapest and are accessible to the public on request from the competent Depositing Authority.

The probiotic bacteria can be in solid form, in particular in the form of powder, dehydrated powder or lyophilized powder.

The oily suspension of the present invention is prepared according to techniques known to experts in the field.

In practice, a determinate quantity of oil is introduced into a container provided with stirring and heating means. Subsequently, the probiotic bacteria in solid form are added gradually, under stirring, avoiding the formation of lumps and agglomerates. When the addition of the bacteria is completed, the oily suspension is kept stirred for a time of between 1 and 30 minutes, possibly by means of gentle heating to a temperature of between 25° C. and 40° C., preferably between 30° C. and 35° C.

In a preferred embodiment of the present invention, the probiotic bacteria can be utilized in micro-encapsulated form, i.e. coated with a composition containing at least one lipid, preferably of vegetable origin. The micro-encapsulated bacteria are then added to the oil, with the same operative procedures as stated above.

In another embodiment of the present invention, the bacteria added to the oil can be in the form of micro-encapsulated bacteria and non-micro-encapsulated “naked” bacteria.

In a preferred embodiment, the probiotic bacteria are coated with a single coating of vegetable origin.

In another preferred embodiment, the probiotic micro-organisms are coated with a first and a second coating of vegetable origin.

In a preferred embodiment, lipids of vegetable nature are selected from the group comprising the saturated vegetable fats having a melting point between 35° C. and 75° C., preferably between 45° C. and 65° C., advantageously between 50° C. and 60° C.

In a preferred embodiment, saturated vegetable fats with a certain degree of hydrophilicity can be used, which can be selected from among the mono- and di-glyceride of saturated fatty acids, the esterified polyglycerols with saturated fatty acids and the free saturated fatty acids.

The saturated fatty acids can be selected from the group comprising between 8 and 32 carbon atoms, preferably between 12 and 28 carbon atoms, even more preferably between 16 and 24 carbon atoms.

Advantageously, the coating lipid is selected from the group comprising polyglyceryl distearate (commercial name Plurol Stearique WL 1009), glyceryl palmitostearate (commercial name Precirol Ato 5), saturated fatty acids (commercial name Revel C), hydrogenated vegetable fats of non-lauric origin and hydrogenated palm fats or stearin.

In a first embodiment, the probiotic bacteria are coated with a single coating (mono-coated). In practice, a single coating with the same lipid is carried out.

Advantageously, the single coating consists of polyglyceryl distearate or polyglycerol ester of vegetable origin or polyglyceryl-6-distearate CAS 61725-93-7 (commercial name Plurol Stearique WL1009).

In a preferred embodiment, the ratio by weight between lyophilized micro-organism and the lipid coating substance which coats them is 50:50 or 40:60.

In a second preferred embodiment, the probiotic bacteria are double-coated. In practice, a double coating is carried out, in succession, with two lipids different from each other (double coating: a first and a second coating separate from each other).

Advantageously, the two lipids are selected from the group comprising a hydrogenated palm fat (Tm=60° C.) and a glycerol dipalmitostearate (Tm=57-60° C.). The two lipids are sprayed onto the lyophilized bacteria in succession, i.e. a double covering is applied to the lyophilate: the first with the hydrogenated palm fat and the second with the glycerol dipalmitostearate in the ratio 3:1 to each other.

The bacteria, preferably in micro-encapsulated form, can be micro-encapsulated using the ordinary techniques known to experts in the field. For example, a fluid bed technique can be used (for example, top-spray or bottom-spray), in which coating materials of a lipid nature are used.

In a first embodiment, two lipids selected from between a hydrogenated palm fat (Tm=60° C.) and a glycerol dipalmitostearate (Tm=57-60° C.) are sprayed onto the lyophilate in succession, i.e. a double covering is applied to the lyophilate: the first with the hydrogenated palm fat and the second with the glycerol dipalmitostearate in the ratio 3:1 to each other. A double coating of the cells ensures better sealing of the bacteria from the environment, producing a continuous film without pores communicating with the outside. This wrapper, however, must open at intestinal level to release the bacteria and allow them to colonize. The lipids selected are in fact resistant to acid pH, so that the coating remains intact in the stomach, but sensitive to even slightly basic pH, so as to allow the formation of holes in the coating during its passage through the intestine.

The oily suspension contains the bacteria in a quantity less than or equal to 30% by weight, between 0.05% and 20% by weight, compared with the total weight of the suspension; preferably in a quantity of between 0.5% and 10%; even more preferably in a quantity of between 1.5% and 5% by weight, compared with the total weight of the suspension.

The oily suspension comprises at least one oil, edible and suitable for being administered to patients of paediatric age, said oil being selected from the group comprising: olive oil, maize oil, soya oil, linseed oil, groundnut oil, sesame oil, fish oil and rice oil.

Advantageously, said oils are of biological grade and can include in their preparation a refining stage and/or a cold pressing stage.

The oily suspension comprises at least one oil in a quantity greater than or equal to 70% by weight, compared with the total weight of the suspension, preferably in a quantity of between 75% and 95% by weight, advantageously at least 90% by weight.

Advantageously, the oily suspension contains only olive oil or olive oil mixed with maize oil and/or soya oil and/or linseed oil. Advantageously, the olive oil is extravirgin and of Bio grade.

In a preferred embodiment, the oily suspension comprises furthermore at least one finely-divided food compound, selected from the group comprising silica, silicon dioxide, silica gel, colloidal silica, precipitated silica, syloid® 244, talc, magnesium silicate, magnesium oxide, magnesium carbonate, calcium silicate, lecithin, mono- or di-glycerids such as glyceril monostearate, glyceril monooleate, plurololeic acid, starch, modified starches, Konjac gum, xanthan gum, gellan gum, carrageenan.

Said material is present in a quantity of between 0.1% and 15% by weight, compared with the total weight of the suspension, preferably of between 5% and 10% by weight, compared with the total weight of the suspension.

In this case the preparation procedure provides that a determinate quantity of oil has added to it the finely-divided food material, for example silicon dioxide, under stirring and heating the oil to about 60° C., until it is completely dissolved.

Alternatively, the silicon dioxide can be added cold; dissolving will however require more time.

Subsequently, the suspension is allowed to cool from 60° C. to room temperature. Next, the lyophilate is weighed and added to the suspension under stirring, until complete and homogeneous dispersion is achieved.

An example of a suspension comprising oil, probiotic bacteria and a finely-divided material is shown in Table 2.

In a preferred embodiment, the oily suspension can furthermore comprise at least one prebiotic fibre and/or at least one carbohydrate with bifidogenic action. The prebiotic fibres and the carbohydrates have a double function. The first is to serve a prebiotic purpose. The second is to serve a technological purpose as a thickener and stabilizer.

The prebiotic fibres and carbohydrates are selected for example from among inulin, fructo-oligosaccharides (FOS), galacto- and transgalacto-oligosaccharides (GOS and TOS), gluco-oligosaccharides (GOSα), xylo-oligosaccharides (XOS), chitosan-oligosaccharides (COS), soya-oligosaccharides (SOS), isomalto-oligosaccharides (IMOS), maltodextrin, resistant starch, pectin, psyllium, arabino-galactanes, gluco-mannanes, galacto-mannanes, xylanes, lactosaccharose, lactulose, lactitol and various other types of gums, acacia fibre, carruba fibre, oat fibre, bamboo fibre, citrus fibres and, in general, fibres containing a soluble portion and an insoluble portion, in variable ratios to each other.

In a preferred embodiment of the invention, said prebiotic fibres and carbohydrates are selected from among gluco-oligosaccharides (GOSα), fructo-oligosaccharides (FOS), inulin and/or maltodextrin.

In a preferred embodiment of the invention, the composition comprises at least one prebiotic fibre selected from among those mentioned above and/or suitable mixtures between them in any relative percentage.

The quantity of prebiotic fibres and/or of carbohydrates with bifidogenic action, if present, is between 0.5% and 25% by weight, preferably between 1% and 20% and even more preferably between 5% and 10%, compared with the total weight of the suspension. In this case the result is a suspension with symbiotic activity.

Furthermore, the suspension can also comprise other active ingredients and/or components such as vitamins, minerals, bioactive peptides, substances with anti-oxidizing action, hypocholesterolaemic agent, hypoglycaemic agent, anti-inflammatory and anti-sweetening agents in a quantity generally of between 0.001% and 10% by weight, preferably between 0.5% and 5% by weight, always depending on the type of active component and its recommended daily dose if any, compared with the total weight of the suspension.

An object of the present invention is the oily suspension for use as a medicament for the treatment of intestinal disturbances such as for example colic in paediatric patients.

A further object of the present invention is the use of said strains of micro-organisms for the preparation of a medicament or supplement for the treatment of certain intestinal disturbances such as for example colic in paediatric patients.

The Applicant has tested the decay of the bacterial load in the following oils: Olive oil, maize oil, cold-pressed soya oil and cold-pressed linseed oil.

The strain used was LMG P-21380 (Lactobacillus paracasei LPC 00), in lyophilized form, having an initial load of 400 MLD/g. A mixture of the pure lyophilate in oil was prepared so as to have 1 MLD/10 ml of oil (2.5 mg in 10 ml of oil). The results are reported in Table 3. Table 3 shows: 1) Bio olive oil, 2) maize oil, 3) sunflower oil, 4) cold-pressed soya oil, 5) cold-pressed linseed oil.

T0=Start time, E-05=1×10⁻⁵, E-09=1×10⁻⁹, E-14=1×10⁻¹⁴, Bn=Billion

1. An oily suspension, particularly for paediatric use, comprising:
at least one food oil selected from: olive oil, maize oil, soya oil, linseed oil, groundnut oil, sesame oil, fish oil and rice oil, said at least one oil being present in a quantity greater than or equal to 70% by weight of the suspension, and
at least one strain of micro-organism selected from the following species: L. acidophilus, L. crispatus, L. gasseri, L. delbrueckii group, L. salivarius, L. casei, L. paracasei, L. plantarum group, L. rhamnosus, L. reuteri, L. brevis, L. buchneri, L. fermentum, B. adolescentis, B. angulatum, B. bifidum, B. breve, B. catenulatum, B. infantis, B. lactis, B. longum, B. pseudocatenulatum and S. thermophilus, wherein said strain is present in a quantity less than or equal to 30% by weight, compared with the total weight of the suspension, and
wherein said micro-organism is coated with at least one coating comprising at least one vegetable lipid having a melting point between 35° C. and 75° C.

2. The suspension according to claim 1, wherein the oil consists of olive oil only.

3. The suspension according to claim 1, wherein said oily suspension further comprises, in a quantity of between 0.1% and 15% by weight of the suspension, at least one finely divided food compound selected from silica, silicon dioxide, silica gel, colloidal silica, precipitated silica, talc, magnesium silicate, magnesium oxide, magnesium carbonate, calcium silicate, lecithin, mono- or di-glycerids, glyceril monostearate, glyceril monooleate, plurol-oleic acid, starch, modified starches, Konjac gum, xanthan gum, gellan gum, and carrageenan.

4. The suspension according to claim 1, wherein said oily suspension furthermore further comprises, in a quantity of between 0.5% and 25% by weight, of the suspension, at least one prebiotic fibre and/or at least one bifidogenic carbohyrate selected from inulin, fructo-oligosaccharides (FOS), galacto- and transgalacto-oligosaccharides, (GOS and TOS), gluco-oligosaccharides (GOSα), xylo-oligosaccharides (XOS), chitosan-oligosaccharides (COS), soya-oligosaccharides (SOS) isomalto-oligosaccharides (IMOS), maltodextrin, resistant starch, pectin, psyllium, arabino-galactanes, gluco-mannanes, galacto-mannanes, xylanes, lactosaccharose, lactulose, lactitol, acacia fibre, carruba fibre, oat fibre, bamboo fibre and citrus fibre.

5. The suspension according to claim 4, wherein said at least one fibre and said at least one carbohyrate are selected from gluco-oligosaccharides (GOSα), fructo-oligosaccharides (FOS), inulin, maltodextrin and mixtures thereof.

6. The suspension according to claim 1, wherein said vegetable lipid has a melting point comprised from 45° C. to 65° C.

7. The suspension according to claim 6, wherein said vegetable lipid has a melting point comprised from 50° C. to 60° C.

8. The suspension according to claim 1, wherein said strain of micro-organism is coated with a single lipid coating.

9. The suspension according to claim 8, wherein said lipid coating consists of polyglyceryl-6.

10. The suspension according to claim 1, wherein said strain of micro-organism is coated with a first lipid coating and a second lipid coating.

11. The suspension according to claim 10, wherein the first lipid coating consists of a hydrogenated palm fat and the second lipid coating consists of a glycerol dipalmitostearate.

12-17. (canceled)

18. The suspension according to claim 9, wherein the ratio by weight of micro-organisms:lipid coating is 50:50 or 40:60.

19. The suspension according to claim 11, wherein the ratio by weight of first lipid coating:second lipid coating is 3:1.

20. The suspension according to claim 1, wherein the oil consists of a mixture of olive oil with maize oil, soya oil, linseed oil or mixtures thereof.

21. A method for treating an intestinal disturbance in a subject, the method comprising administering to a subject in need thereof, a suspension in accordance with claim 1, such that the intestinal disturbance is treated.

22. The method according to claim 21, wherein the intestinal disturbance is colic in a paediatric subject.

23. The method according to claim 21, wherein the micro-organism is coated with a single lipid coating.

24. The method according to claim 23, wherein the lipid coating consists of polyglyceryl-6-distearate.

25. The method according to claim 21, wherein the strain of micro-organism is coated with a first lipid coating and a second lipid coating.

26. The method according to claim 25, wherein said first liquid coating consists of a hydrogenated palm fat and the second lipid coating consists of a glycerol dipalmitostearate.
